(19) United States
(12) Patent Application Publication
(10) Pub. No.: US 2007/0190141 A1
(43) Pub. Date: Aug. 16, 2007

(54) Extended release opiate composition

(76) Inventor: Aaron Dely, Lighthouse Point, FL (US)

Correspondence Address: HEDMAN & COSTIGAN P.C., 1185 AVENUE OF THE AMERICAS, NEW YORK, NY, 10036, US

(21) Appl. No.: 11/706,579

(22) Filed: Feb. 15, 2007

Related U.S. Application Data
(60) Provisional application No. 60/773,874, filed on Feb. 16, 2006.

(51) Int. Cl. A61K 9/22

(52) U.S. Cl. 424/468

(57) ABSTRACT

The present invention relates to an extended release opiate pellets that contain an opiate and an inorganic osmagent in the core. The extended release pellets can be used alone or combined with an immediate release opiate form to prepare extended release oral pharmaceutical tablets or capsules.

### CROSS REFERENCE TO RELATED APPLICATION

This application claims the benefit of provisional patent application Ser. No. 60/773,874 filed on Feb. 16, 2006.

### FIELD OF THE INVENTION

The present invention relates to the field of oral dosage forms. More particularly, it relates to an extended release oral dosage form of an opiate for the treatment in mammals of pain arid other related disorders.

### BACKGROUND OF THE INVENTION

Opiates have been used to treat pain in humans for millennia. Many opiates are well known in the art. A preferred embodiment of the present invention pertains to extended release composition comprising morphine, and more specifically, the sulfate salt of morphine. Morphine has the molecular formula C₁₇H₁₉NO₃; and a molecular weight of 285.34 daltons (both in the base form) (see Merck Index p. 1121; entry 6300, 13^{th} Ed. (2001) and Physicians' Desk Reference 1815-17, 59^{th} Ed. (2005)).

The drug morphine sulphate is commercially available in capsules, injections, and oral solutions. Extended release capsule formulations are also commercially available and sold under the tradenames AVINZA® and KADIAN®. See Physician Desk Reference pp. 1815 and 569 (59^{th} Ed. 2005) (which is incorporated herein by reference). Due to the nature of opiates such as morphine sulfate, it is often difficult to obtain 100% or nearly 100% release of the opiate from an extended release dosage form while at the same time preventing the opiate from releasing to quickly. The failure in obtaining full release of the opiate results in the patient obtaining less than the required amount of opiate, which in turn can result in unnecessary pain and discomfort.

There exists a need for an orally administrable opiate medication with a high concentration of active ingredient that can be manufactured in an economically feasible manner and which releases nearly all of the opiate in a safe and reliable manner. U.S. Pat. Nos. 5,202,128; 5,378,474 and 6,066,339 (incorporated herein by reference) describes oral pharmaceutical formulations containing multiparticulate and/or pellet formulations comprising morphine sulphate. These formulation are highly complex, time consuming to produce and expensive to manufacture. U.S. Pat. Nos. 5,202,128 and 5,378,474 describe a pelletized formulation that employs a combination of enteric polymers, matrix polymers and compounds soluble in low pHs. These components are combined to form release coatings that produce a substantially zero order release of opiate medication in vivo. U.S. Pat. No. 6,066,339 recites a multiparticulate oral dosage form of morphine sulphate that employs osmotic agents and enteric polymers, specifically ammonia methacrylate copolymers. The above patents all disclose costly and time consuming compositions and methods of manufacturing extended release composition comprising morphine sulphate.

### SUMMARY OF THE INVENTION

The present invention overcomes the drawbacks of the prior art through the novel development of a multiparticulate extended release oral opiate formulation that is simple and inexpensive to manufacture. The substantially reduced manufacturing time and costs are increasingly necessary in the highly competitive marketplace of United States pharmaceuticals.

An embodiment of the present invention involves the formation of an immediate release pellet comprising an opiate, preferably a pharmaceutically acceptable form of morphine such as a sulfate or hydrochloride salt, and an inorganic osmotic agent. The immediate release pellet is then used to form an extended release pellet by coating the immediate release pellets with a controlled release coating that comprises a water-insoluble water permeable film-forming compound, such as a polymer to control the release of the opiate. The release rate of the opiate from the extended release pellet is controlled by the thickness of the coating and the composition of the coating. For example the thicker the film coating, the slower the release of the opiate from the extended release pellet. Similarly, the less porous the film coating, i.e. less pore forming material in the film coating, the slower the release of the opiate from the extended release pellet. The extended release pellets of the present invention may be used alone or combined with an immediate release form of the opiate. The immediate release form of the opiate may be free opiate that is mixed with the extended release pellets, immediate release pellets, opiate that is coated onto the outer surface of the extended release pellets or any other immediate release form commonly known in the industry. The present invention may also employ multiple extended release pellets or a heterogeneous population of extended release pellets. A heterogeneous population of extended release pellets is a group of extended release pellets that exhibit different release profiles due to variations in the thickness of the film coating and/or different composition of the film coating. By varying the amounts of immediate release opiate and extended release pellets the desired in vitro and in vivo release profiles can be obtained. The final dosage form may be a hard or soft gelatin capsule or formed into a tablet using conventional tablet forming techniques. The extended release pellets may also be sprinkled or mixed with food prior to administration for those patients that have difficulty in swallowing.

As used herein, the term “pellet” refers to and includes small discrete units that that are about 1 cm or smaller in size (length or diameter). The term includes granules, spheroids, beads, microspheres, and seeds. It is preferred, but not necessary, that the pellets be spherical or close to spherical in shape.

In a preferred embodiment, the dosage form will be administered once a day, ideally with or after a meal, and provide therapeutic levels of the drug throughout the day with a plasma peak occurring between 4 and 10 hours, followed by a slight drop in the plasma level and a second plasma peak occurring between 10 hours and 16 hours. If an immediate release component is included in the dosage form of the present invention a third plasma peak will be obtained within 2 hours after administration. The usual opiate dosage range for the present invention is 30-200 mg.

One embodiment of the present invention which exhibits all the foregoing advantages is obtained by preparing an extended release pellet comprising:
a) a core or immediate release pellet comprising:
   (i) an opiate; and
   (ii) an inorganic osmagent;

wherein said core or immediate release pellet is further coated with varying levels of
(b) a release controlling composition comprising:
   (i) a water permeable, water insoluble compound;
   (ii) optionally a plasticizer; and
   (iii) optionally a channeling agent.

The present invention also relates to a method of producing the extended release pellets or beads.

### DETAILED DESCRIPTION OF THE INVENTION

The pharmaceutically active ingredient or drug that is used in the present invention is an opiate which includes natural and synthetic opioids as well as the active metabolites such as morphine-6-glucuronide and morphine-3-glucuronide. The term also includes pharmaceutically acceptable salts and/or complexes of opioids. Some examples of opioids are alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, dextropropoxyphene, ketobemidone, levallorphan, levorphanol, levophenacylmorphan, lofentanil, meperidine, meptazinol, metazocine, methadone, metopon, morphine, myrophine, nalbuphine, narceine, nicormorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, and tramadol. In a preferred embodiment the opiate is morphine, and even more particularly morphine sulphate.

The inorganic osmagent that is used in the core of the present invention is an inorganic material that attracts fluid into the core. The preferred inorganic osmagents are inorganic salts preferably, alkali and alkali earth metal salts. Representative examples of the inorganic osmagents that are useful in the present invention include but are not limited to magnesium sulfate, potassium chloride, ammonium chloride, calcium sulfate, sodium chloride, lithium chloride, lithium sulfate, potassium sulfate, sodium sulfate, and the like or combinations thereof. Other inorganic osmagents are described in U.S. Pat. Nos. 4,612,008 and 5,082,668; which are incorporated herein by reference. The preferred osmagent for use in the present invention is sodium chloride.

The core or immediate release pellet of the present invention can be prepared by methods commonly known in the art such as wet granulation, dry granulation or extrusion spheronization. In a preferred embodiment of the present invention, the core or immediate release pellet is prepared with an inert starting seed such as a sugar seed with a mesh size of 15-40, preferably 20-25. If an inert starting seed is employed it must be of sufficient density and strength to enable it to undergo coating with the opiate and the controlled release composition. Suitable starting seeds are sugar seeds or non-pariels that are well known in the art. Additional suitable starting seeds may be chosen from plastic resins, silica, glass, microcrystalline cellulose, starches and other materials commonly known in the art.

The opiate and inorganic osmagent can be applied to the inert starting seed by any conventional techniques known in the industry, such as, pan coating, roto-granulation or fluidized bed coating. During such coating operations the opiate is dispersed or dissolved in a solvent, which may also contain other conventional excipients. Suitable excipients may be, but are not limited to organic osmagents, binding agents, surfactants, plasticizers, glidants, lubricants, stabilizers, pH modifying agents, solubilizers and combinations of the foregoing.

Although the aforementioned excipients are commonly known in the industry, a few examples of the binding agents that may be employed in the present invention are polyvinyl pyrrolidone, hydroxyethyl cellulose, hydroxypropyl cellulose, hydroxypropyl methylcellulose, polyacrylate, ethylcellulose, or mixtures of the foregoing. It is preferred that the binding agent be a water soluble or rapidly water dispersible material such as the low molecular weights polyvinyl pyrrolidone, hydroxpropyl methylcellulose and hydroxpropyl cellulose such as those described in European Patent Application No. 1588708A (which correspondes to WO 04/067001) which is incorporated herein by reference.

Once the cores or immediate release pellets are prepared, a controlled release coating is applied to the cores or immediate release pellets. The controlled release coating preferably comprises a film forming compound such as a polymer and optionally conventional excipients such as a plasticizer and a channeling agent. The film forming polymer is preferably a water permeable, water insoluble polymer. Some examples of the film forming polymer useful in the present invention are cellulose esters, cellulose dieters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770; 3,916,899; 4,008,719 4,036,228 and 5,260,068; which are incorporated herein by reference. The most preferred film forming polymer is cellulose acetate comprising an acetyl content of 37% to 42%, which is commercially available from Eastman Fine Chemicals.

The controlled release coating may also comprise a plasticizer, which makes the extended release coating more flexible and aids in the release of the opiate from the core or immediate release pellet. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. Other possible plasticizers include triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. The preferred plasticizer are water soluble, such as acetyltributyl citrate.

It may also be desirable for the controlled release coating to employ a channeling agent. A channeling agent is a material that increases the volume of fluid imbibed into the core to enable the extended release pellet to dispense substantially all of the opiate through the controlled release coating. The channeling agent may dissolve or leach from the controlled release coating to form paths in the coating for the fluid to enter the core and dissolve the opiate. The dissolved opiate is then released from the extended release pellets by movement through the channels formed in the controlled release coating by the dissolved or leached channeling agent. The channeling agent can be a water soluble material or an enteric material. Some examples of the preferred materials that are useful as channeling agents are sodium chloride, potassium chloride, sucrose, sorbitol, mannitol, polyethylene glycol (PEG), propylene glycol, poloxamer, hydroxypropyl cellulose, hydroxypropyl methycellulose, hydroxypropyl methycellulose phthalate, cellulose acetate phthalate, polyvinyl alcohols, methacrylic acid copolymers and mixtures thereof. The preferred channeling agent is PEG 400.

The channeling agent may also be a drug that is water soluble. If the channeling agent is a drug, the dosage form has the added advantage of providing an immediate release of the drug that is selected as the channeling agent.

The controlled release coating may also further comprise a lubricant such as talc, stearic acid, magnesium stearate, silicon dioxide, glyceryl monostearate, kaolin and sodium stearate. A particularly preferred lubricant is talc. In capsule formulation the pellets may be dusted with a lubricant before encapsulation, whereas during tabletting the lubricant is added before compression and/or during granulation of the material to be tabletted.

The weight percentages of ingredients shown in Table I below are based on a preferred embodiment of the present invention and the total weight of the core or immediate release pellet composition.

TABLE I

| Ingredients (core) | Preferred | Most preferred |
|---|---|---|
| Inert seed | 20-70% | 30-60% |
| Opiate | 10-60% | 15-45% |
| Inorganic osmagent | 1-30% | 2-20% |
| Binder | 0-30% | 1-20% |

The weight percentages of the ingredients shown in Table II are based on a preferred embodiment of the present invention and are based upon the total dry weight of the extended release coating composition.

TABLE II

| Ingredients (coating) | Preferred | Most preferred |
|---|---|---|
| Film forming polymer | 50-99% | 70-90% |
| Plasticizer | 0-40% | 1-35% |
| Channeling agent | 0-20% | 1-10% |
| Lubricant | 0-10% | 0.5-5% |

A final dosage form prepared in accordance with the present invention may contain a homogeneous population of extended release pellets or a heterogeneous population of extended release pellets. As used herein a homogeneous population of extended release pellets refers to extended release pellets that employ a similar controlled release coating composition and thickness. As used herein a heterogeneous population of extended release pellets refers to two or more different types of extended release pellets wherein each type of extended release pellet employs a different amount of the controlled release coating (i.e. thickness) and/or a different controlled release coating composition such as a different film forming material or different amount of channeling agent. A method for preparing a heterogeneous population of extended release pellets is disclosed in U.S. Pat. No. 6,524,620; which is incorporated herein by reference.

An embodiment of the present invention will be a capsule of tablet that contains about 1-40% (preferably 2-20%) of an immediate release form of the opiate, preferably in the form of immediate release pellets; and about 60-98% of extended release pellets. Another embodiment of the present invention will be a capsule of tablet that contains about 1-30% of an immediate release form of the opiate, preferably in the form of immediate release pellets; about 30-90% of a first population of extended release pellets that cause a plasma peak between 4 and 10 hours after administration and 30-90% of a second population of extended release pellets that cause a plasma peak about 10-16 hours after administration. Still another embodiment of the present invention will be a capsule of tablet that contains about 2-20% of an immediate release form of the opiate, preferably in the form of immediate release pellets; about 20-60% of a first population of extended release pellets that exhibits a zero order rate of opiate release over a 24 hour time period and 20-60% of a second population of extended release pellets that exhibits a lag time where little or no opiate is released, independent of pH of the environment of use, followed by a zero order rate of opiate release over a time period of about 2 to about 6 hours following the lag time. The lag time for the second population can be for a period of about 4 to about 14 hours, preferably about 6 to about 12 hours.

### DESCRIPTION OF THE PREFERRED EMBODIMENT

The present invention will be further illustrated by the following examples.

### EXAMPLE I

An extended release morphine sulphate capsule in accordance with the present invention is prepared as follows.

Step I Drug Solution Morphine sulphate, sodium chloride and Opadry® Clear are dissolved into a water solution. The solution is mixed for approximately 60 minutes.

Step II Spray Coating

The suspension formed in Step I above is spray coated onto 20/25 mesh sugar spheres using a fluidized bed coater under standard coating parameters to form immediate release pellets.

Step III Release Controlling Solution

A release controlling solution is formed by dissolving cellulose acetate, acetytributyl citrate and polyethylene glycol in acetone.

Step IV Controlled Release Coating

A portion of the release controlling solution formed in Step III above is sprayed dried onto some of the immediate release pellets formed in Step II to form an extended release pellet that exhibits a zero order rate of opiate release over a 24 hours time period and which results in a plasma peak in about 4 to 10 hours after administration.

Step V Controlled Release Coating (lag time)

A second portion of the release controlling solution prepared in Step III above is sprayed onto an additional sample of the immediate release pellets formed in Step II above to form an extended release pellet that exhibits a lag time of about 6 to about 12 hours after administration wherein no or substantially no opiate is released. The extended release lag time pellets will begin releasing the opiate after the lag time in a substantially zero order rate for a period of about 2 to about 6 hours resulting in a plasma peak of about 10-16 hours after administration.

Step VI Encapsulation

The immediate release and extended release pellets prepared above are dusted with talc and encapsulated utilizing equipment and guidelines commonly known in the art. Final capsules are prepared comprising from about 2-20% of the immediate release pellets formed from Step II; from about 20-60% of the extended release pellets from Step IV and from about 10-50% of the extended release pellets from Step V.

The composition of the final dosage form is report in Table III.

TABLE III

| | Amount |
|---|---|
| Ingredients | per Unit |
| Morphine Sulfate USP | 120.0 |
| Sugar Spheres, NF Pg 20/25 | 160.0 |
| Opadry Clear (YS-1-7006) | 42.0 |
| Sodium Chloride, USP | 30.0 |
| Cellulose Acetate, NF 398-10 | 34.5 |
| Acetyltributyl Citrate, NF | 5.8 |
| Polyethylene Glycol 400 | 1.2 |
| Talc | 20.7 |
| Purified Water, USP | * |
| Acetone, NF | * |
| Total | 414.1 |

* evaporated during processing.

While certain preferred and alternative embodiments of the invention have been set forth for purposes of disclosing the invention, modifications to the disclosed embodiments may occur to those who are skilled in the art. Accordingly, this specification is intended to cover all embodiments of the invention and modifications thereof which do not depart from the spirit and scope of the invention.

We claim:

1. An extended release opiate pellet comprising a core and a controlled release coating wherein:
the core comprises:
(i) an opiate; and
(ii) an inorganic osmagent;
and a controlled release coating surrounds the core and comprises:
(i) a water permeable, water insoluble film forming compound;
(ii) optionally a plasticizer; and
(iii) optionally a channeling agent.

2. The extended release opiate pellet according to claim 1, wherein said opiate is morphine sulphate.

3. The extended release opiate pellets according to claim 1, wherein the core further comprises inert starting seed.

4. The extended release opiate pellet according to claim 1, wherein the water-permeable water insoluble film forming compound is cellulose acetate.

5. The extended release opiate pellet according to claim 1, wherein the inorganic osmagent is sodium chloride.

6. An oral pharmaceutical dosage form comprising the extended release pellet of claim 1.

7. An oral pharmaceutical dosage form as defined in claim 6 wherein the dosage form is a capsule.

8. An oral pharmaceutical dosage form as defined in claim 6 wherein the dosage form is a tablet.

9. An oral pharmaceutical dosage form as defined in claim 6 wherein the dosage form further comprises an immediate release form of the opiate.

10. An oral pharmaceutical dosage form comprising an immediate release form of an opiate and a plurality of extended release opiate pellets wherein the extended release opiate pellets comprise a core and a controlled release coating wherein the core of the extended release pellets comprises:
(i) an opiate; and
(ii) an inorganic osmagent;
and a controlled release coating on the extended release pellets surrounds the core and comprises:
(i) a water permeable, water insoluble film forming compound;
(ii) optionally a plasticizer; and
(iii) optionally a channeling agent.

11. The oral pharmaceutical dosage form as defined in claim 10 wherein the extended release opiate pellets are a homogeneous population of pellets.

12. The oral pharmaceutical dosage form as defined in claim 11 wherein the extended release opiate pellets are a heterogeneous population of pellets.
